(19) United States
(12) Patent Application Publication
(10) Pub. No.: US 2025/0213852 A1
(43) Pub. Date: Jul. 3, 2025

(54) Wearable Holding Device for Neural Implant Headpiece

(71) Applicant: Wyss Center for Bio and Neuro Engineering, Genève (CH)

(72) Inventor: Ulysse Martel, Lausanne (CH)

(21) Appl. No.: 18/851,207

(22) PCT Filed: Apr. 10, 2023
(86) PCT No.: PCT/IB2023/053646

§ 371 Date: Sep. 26, 2024

Related U.S. Application Data
(60) Provisional application No. 63/362,794, filed on Apr. 11, 2022.

(51) Int. Cl. A61N 1/05

(52) U.S. Cl. CPC A61N 1/0526

(57) ABSTRACT

A wearable holding device for a neural implant headpiece prevents disconnection or misalignment and is comfortable to wear during sleep or during other regular or long-term use at any time of day. The wearable holding device comprises at least one pad surrounding at least part of the neural implant head-piece, and configured to be mounted to an exterior of a head. The at least one pad forms a cavity configured to receive and cover the at least part of the neural implant head-piece and to continuously maintain stability of the neural implant head-piece relative to the head. A holding textile is configured to hold the at least one pad forming the cavity to the head.

### RELATED APPLICATION

This application claims the benefit of U.S. Provisional Application No. 63/362,794, filed on Apr. 11, 2022. The entire teachings of the above application are incorporated herein by reference.

### BACKGROUND

Ongoing developments in the field of neural implants aim to provide batteryless or rechargeable neural implants that can be powered or re-charged using a head-piece. Such a head-piece can be mounted to the head, for example using magnets or adhesives, but could potentially suffer from many drawbacks in regular use. For example, the head-piece could become disconnected during sleep, or make sleep uncomfortable. The neural implant could become misaligned, tear off or fall off, have its cable dangle from the device or become entangled, be of an annoying weight or size, cause skin pressure or skin erosion, or become humid. There is also a need for neural implants to be held in place and prevented from motion, such as to be prevented from motion relative to a wearer's head.

### SUMMARY

Embodiments provide a wearable holding device for a neural implant headpiece, which aim to prevent any of several potential drawbacks of head-pieces, such as the above, for example by preventing disconnection or misalignment during sleep and by being comfortable to wear during sleep, or during other regular or long-term use at any time of day.

In one embodiment, a wearable holding device for a neural implant head-piece comprises at least one pad surrounding at least part of the neural implant head-piece, and configured to be mounted to an exterior of a head. The at least one pad forms a cavity configured to receive and cover the at least part of the neural implant head-piece and to continuously maintain stability of the neural implant head-piece relative to the head. A holding textile is configured to hold the at least one pad forming the cavity to the head.

In further, related embodiments, the at least part of the neural implant head-piece may be configured to communicate at least one of power and data to or from at least one of a power source and a data source, or may comprise a battery, or both. The at least one pad may be configured to deform in shape to follow the topography of the head, and may comprise a plurality of padding mesas extending to a height from a surface of the at least one pad, and separated from each other by padding gaps configured to permit the plurality of padding mesas to be moved closer to each other or further away from each other as the at least one pad is deformed by pressure on the head. The cavity configured to receive and cover the at least a part of the neural implant head-piece may be formed by at least some of the plurality of padding mesas, and the surface of the at least one pad. The at least part of the neural implant head-piece may be configured to communicate at least one of power and data to or from at least one of a power source and a data source, and the at least one pad may form a cord opening for at least one cord carrying the at least one of power and data to or from the at least one of the power source and the data source. The cord opening may be formed by at least some of the plurality of padding mesas. The at least one pad may form an opening for an ear of the head. The cavity configured to receive and cover the at least a part of the neural implant head-piece may be formed to be positioned over at least one of a parietal bone of the head, a temporal bone of the head, a mastoid process of the head, and an occipital bone of the head, when the holding textile holds the at least one pad forming the cavity to the head.

In other related embodiments, the at least one pad may form at least one textile opening configured to permit attaching of the holding textile to the at least one pad. The holding textile may comprise at least one of: a neck strap, a strap configured to extend over a top portion of the head, a forehead strap, and a strap for a back of the head. The holding textile may comprise the neck strap, the strap configured to extend over the top portion of the head, the forehead strap, and the strap for a back of the head. The at least one pad may comprise at least one elongated arm configured to permit attachment to at least one of the neck strap, the strap configured to extend over the top portion of the head, the forehead strap, and the strap for the back of the head. The holding textile may form an opening for an ear of the head.

In further related embodiments, the neural implant head-piece may be configured to be magnetically mounted to the exterior of the head, and the cavity configured to receive and cover the at least a part of the neural implant head-piece may be formed to comprise a surrounding clearance between the at least one pad and the neural implant head-piece that is no larger than a distance of magnetic misalignment of the neural implant head-piece. A surrounding clearance between the at least one pad and the neural implant head-piece may comprise 5 mm or less of clearance between the at least one pad and a side perimeter of the neural implant head-piece. The surrounding clearance may comprise 12 mm or less of height clearance between a top portion of the neural implant head-piece and the at least one pad. The at least one pad may comprise a breathable foam, and may comprise a closed cell foam, such as at least one of an ethylene-vinyl acetate (EVA) foam and a polyurethane foam. The holding textile may comprise a webbing material.

The present disclosure also provides a method of wearing a neural implant head-piece including surrounding at least part of the neural implant head-piece with at least one pad of a wearable holding device. The at least part of the neural implant head-piece being mounted to an exterior of a head. The at least one pad forming a cavity receiving and covering the at least part of the neural implant head-piece and to continuously maintain stability of the neural implant head-piece relative to the head. The at least one pad forming a cavity to the head can be held with a holding textile.

In particular embodiments, the shape of the at least one pad can be deformed to follow the topography of the head. The at least one pad can include a plurality of padding mesas extending to a height from a surface of the at least one pad. The plurality of padding mesas can be separated from each other by padding gaps configured to permit the plurality of padding mesas to be moved closer to each other or further apart from each other as the at least one pad is deformed by pressure on the head. The cavity can be configured to receive and cover the at least part of the neural implant head-piece and can be formed by at least some of the plurality of padding mesas, and the surface of the at least one pad. The at least one pad can form an opening for an ear of the head. The method can further include the details described above for the wearable holding device.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic diagram showing a pad of a wearable holding device for a neural implant head-piece in accordance with an embodiment of the disclosure.

FIG. 2 is a schematic diagram showing a wearable holding device for a neural implant head-piece in accordance with an embodiment of the disclosure.

FIG. 3 is a schematic diagram showing the pad of the embodiment of FIG. 1, surrounding at least part of the neural implant head-piece, in accordance with an embodiment of the disclosure.

FIG. 4 is a schematic diagram showing a neural implant head-piece in accordance with an embodiment of the disclosure.

FIG. 5 is a schematic diagram showing a holding textile, as worn on a head of a wearer, in accordance with an embodiment of the disclosure.

FIG. 6 is a schematic diagram showing the pad of the embodiment of FIG. 1, as worn on a head of a wearer, in accordance with an embodiment of the disclosure.

FIG. 7 is a schematic diagram showing positioning of a neural implant headpiece, on a head of a wearer, in accordance with an embodiment of the disclosure.

### DETAILED DESCRIPTION

A description of example embodiments follows.

A wearable headgear holding device for a neural implant head-piece taught herein helps to prevent disconnection or misalignment of the head-piece during sleep, and is comfortable to wear during sleep, or during other regular or long-term use at any time of day. Misalignment or disconnection of neural implant head-pieces can affect efficiency of data transmission from the neural implant to the head-piece, and can affect power transfer from the head-piece to the implant, or both. At the same time, neural implant head-pieces can potentially create friction, pressure, and tissue damage against the head. The wearable holding device alleviates such potential issues by allowing support and comfort for the head-piece, using padding that covers the head-piece to avoid placing pressure on the user's head and to avoid having the headpiece be ripped off the user's head by movement during sleep.

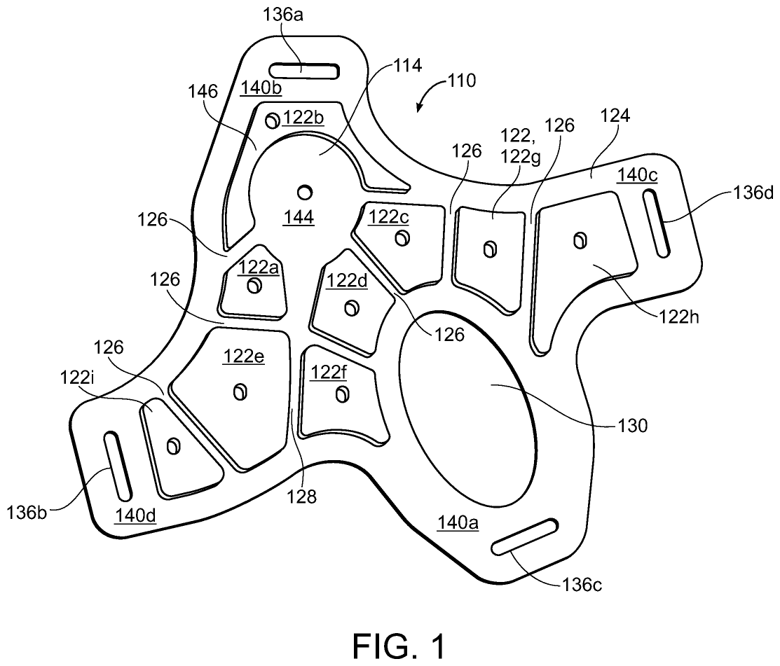

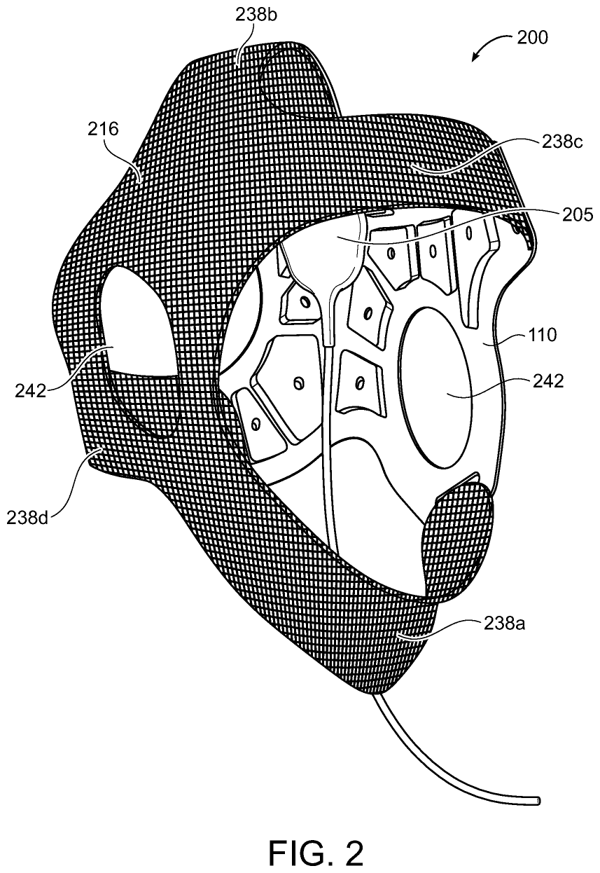

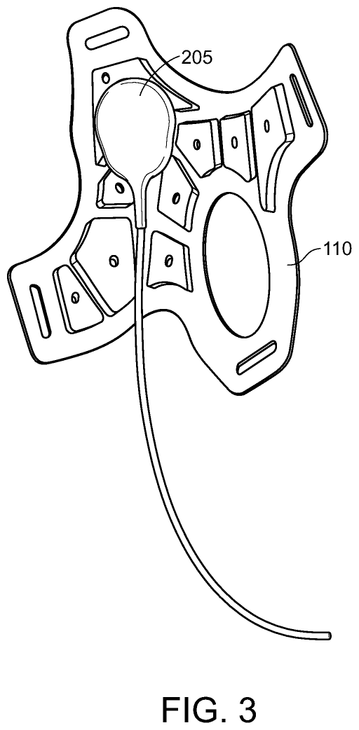

FIGS. 1-3 show the wearable headgear holding device 200 (FIG. 2), at least one pad or pad portion 110 of the device in isolation (FIG. 1), and a partial view of the pad portion 110 surrounding the neural implant head-piece 205 (FIGS. 2 and 3), in accordance with an embodiment of the disclosure. With reference to FIGS. 1-3, the wearable holding device 200 (FIG. 2) for the neural implant head-piece 205 (FIGS. 2 and 3) comprises an inner pad portion or at least one pad 110 (FIGS. 1-3), surrounding at least part of the neural implant head-piece 205 (FIGS. 2 and 3), and configured to be mounted to or against an exterior of a head, for example on the left side. The at least one pad 110 forms a cavity 114 (FIG. 1) configured to receive and cover the at least part of the neural implant head-piece 205 (FIGS. 2 and 3) and to continuously maintain stability of the neural implant head-piece 205 relative to the head, such as on the side of the head above and behind the ear. A flexible headgear or strap portion comprising a holding, fastening or securing material, fabric or textile 216 (FIG. 2) can form an outer securement portion that is configured to hold an inner portion that can include the at least one pad 110 and the cavity 114 (FIG. 1) containing the neural implant head-piece 205 to the head. Embodiments of the at least one pad 110 can fit on and extend on the side of the head around the ear, and extend upwardly, frontwardly and rearwardly relative to the ear.

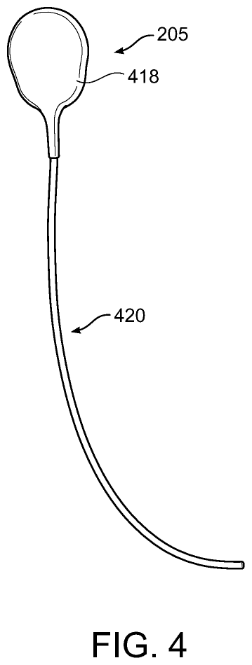

With reference to FIG. 1, the pad portion or the at least one pad 110 can be made to fit a majority of human heads, for comfort, and can be configured to deform in shape to follow the topography of the head. For example, the pad portion 110 can be segmented and comprise a plurality of thickened or raised padding portions, segments or mesas 122 such as 122a-122i, extending to a height from a flexible base layer or surface 124 of the at least one pad 110 on the inner facing side or the inner face. The mesas 122a-122i can have equal heights or thicknesses with flat upper or top surfaces, and can be separated from each other by narrow elongate grooves, channels or padding gaps 126 configured to permit the plurality of padding mesas 122a-122i to be moved closer to each other or further away from each other as the at least one pad 110 and the base surface 124 is deformed or bent by pressure on the head to conform to the rounded shape of the head. The cavity 114 can be configured to receive and cover the at least a part of the neural implant head-piece 205, and can be formed by or defined by lateral edge surfaces of at least some of the plurality of padding mesas such as mesas 122a-122d, as well as an area of base surface 124 that forms an outer sidewall for cavity 114. The inner surface, inner facing side or inner face of the base surface 124 within the cavity 114 can hold or press the neural implant head-piece 205 against the head, or position the head-piece 205 close to the head. The at least one pad 110 can form a downwardly extending cord opening 128 for at least one cord 420 (FIG. 4) extending across arm 140d. The at least one cord 420 can carry at least one of power and data to or from a power source or data source or both. The cord opening 128 can be formed by or defined by opposing lateral edge surfaces of at least some of the plurality of padding mesas 122 such as mesas 122a, 122d, 122e, 122f, extending therepast. The at least one pad 110 can form a hole or opening 130 for an ear of the head extending through the base surface 124. The opening can be generally oval shaped.

The pad portion or the at least one pad 110 and base layer 124, can have four elongate arms 140a-140d, with arm 140a being a lower arm having the opening 130 for the ear, and positioned generally opposite to upper arm 140b. Upper arm 140b can include the cavity 114 for containing the neural implant head-piece 205. Arm 140c can be a front arm that is positioned generally opposite to back arm 140d. The gaps 126 between the padding mesas 122a-122i can extend in a transverse manner across arms 140a-140d to allow the base surface 124 to bend or deform easily between the mesas 122 along the gaps 126 in transverse directions to arms 140a-140d to allow the arms 140-140d to bend and conform to the user's head. The padding mesas 122a-122i and gaps 126 can be mostly positioned on respective arms 140b-140d, and arm 140a can be mostly formed by base layer 124. Bending of arm 140b can be assisted by the transverse gaps 126 between mesa 122b and mesas 122a/122c. Bending of arm 140c can be assisted by the transverse gaps 126 between mesas 122c, 122g and 122h. Bending of arm 140d can be assisted by transverse gaps 126 between mesas 122i and 122e, as well as between mesas 122e/122f and 122a/122d. The opening 130 for the ear can extend from arm 140a into arm 140c, and the upper portion of opening 130 can be adjacent to lateral edges of mesas 122c, 122d, 122f, 122g and 122h. The mesas 122a-122i can have different lateral shapes and dimensions to encircle cavity 114 and the opening 130 for the ear, as well as to provide the cord opening 128 and transverse gaps 126 along arms 140a-140d. The gaps 126 between the mesas 122 can allow opposite arms 140a/140b and 140c/140d to bend inwardly towards each other in a curved segmented manner. When arms 140a-140d bend towards each other, certain gaps 126 can narrow between certain mesas 122, moving these mesas 122 closer together.

FIG. 4 shows the neural implant head-piece 205 in isolation. In embodiments according to the disclosure, the at least part of the neural implant head-piece 205 can be configured to communicate at least one of power and data to or from at least one of a power source (not shown, such as a source of AC or DC power, or both) and a data source (not shown); or can comprise a battery; or both. For example, a body 418 of the neural implant head-piece 205 can comprise a battery inside (not shown), which may be rechargeable using power through a cord 420. Alternatively, the body 418 may contain no battery, and the head-piece 205 may be batteryless. The cord 420 may carry data only, power only, or both data and power. The cord 420 may carry power (along with data or only power), which may be to recharge a battery, to power the neural implant head-piece 205 without a battery, to perform both recharging and sole-powering of the head-piece 205 at different times, or to power some components of the head-piece 205 directly while also recharging a battery of the head-piece. The cord 420 can, for example, be used in a variety of different ways, for example: with a cable clip or an ear clip; and extending from the head-piece to a recorder device or wearable data processing and/or storage device, such as a portable smart phone or other device (not shown), which can be mounted in a sleeve or armband, or on a belt, or can be mounted or clipped to a side of a bed, mattress, or board. The head-piece can also communicate wirelessly with one or more devices, such as a recorder device.

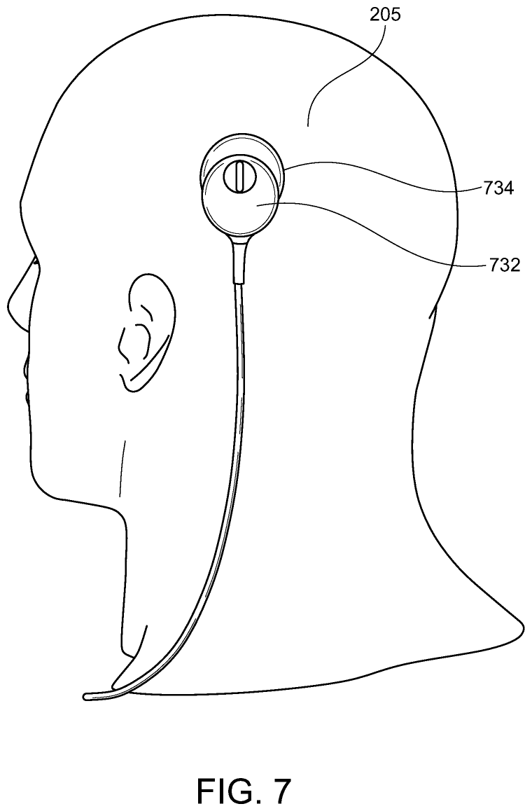

Returning to FIG. 1, the cavity 114 configured to receive and cover the at least a part of the neural implant head-piece 205 can be formed to be positioned over at least one of a parietal bone of the head, a temporal bone of the head, a mastoid process of the head, and an occipital bone of the head, when the holding textile 216 holds the at least one pad 110 forming the cavity 114 to the head. For example, when positioned over the head and holding at least part of the neural implant head-piece 205, the cavity 114 and the base surface 124 portion of the cavity 114 can hold and position the head-piece 205 as shown in FIG. 7, which shows positioning over the parietal bone of the head. In addition, FIG. 7 shows that a rear portion 732, or other portion, of the neural implant head-piece may be shaped to fit at least partly within a pocket or other opening within the at least one pad 110 (FIG. 1), such as in cavity 114 (FIG. 1), while another portion 734 (FIG. 7) of the neural implant head-piece 205 may extend outside of cavity 114 (FIG. 1). Alternatively, all of the head-piece 205 can be contained within the cavity 114. Returning to FIG. 1, the at least one pad 110 can form at least one textile slot or opening 136a-136d in distal ends of arms 140a-140d, that are configured to permit attaching of a holding textile 216 (FIG. 2) to the at least one pad 110, for example using straps (not shown) of the holding textile 216 that extend through the textile openings 136a-136d (FIG. 1) and attach back to the holding textile 216 (for example using a Velcro® or other hook and loop attachment, a clasp, button, clip, sewing, or other textile attachment). The at least one pad 110 (FIG. 1) can be made of, or include, a soft, comfortable, washable, and breathable foam, such as a closed cell foam, for example ethylene-vinyl acetate (EVA) foam, polyurethane foam, or both. In other embodiments, the pad portion or the at least one pad 110 can be held within a pocket of the holding textile 216.

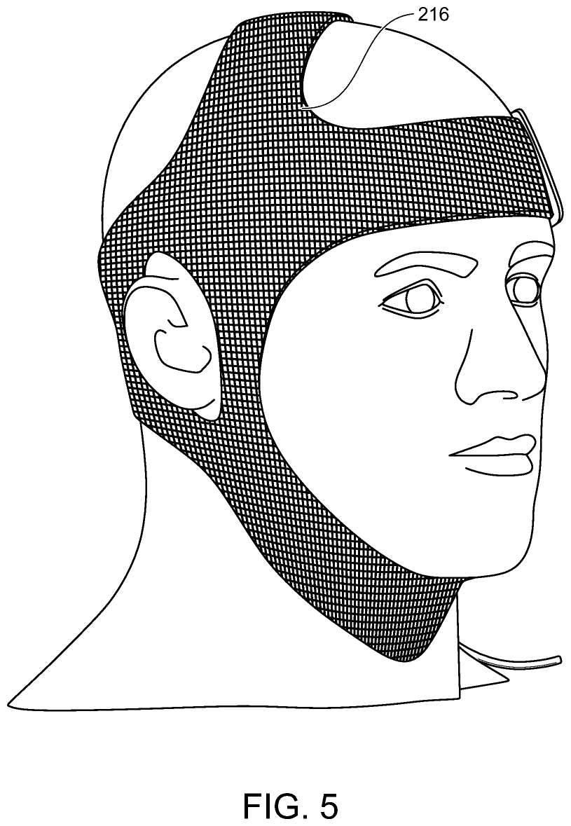

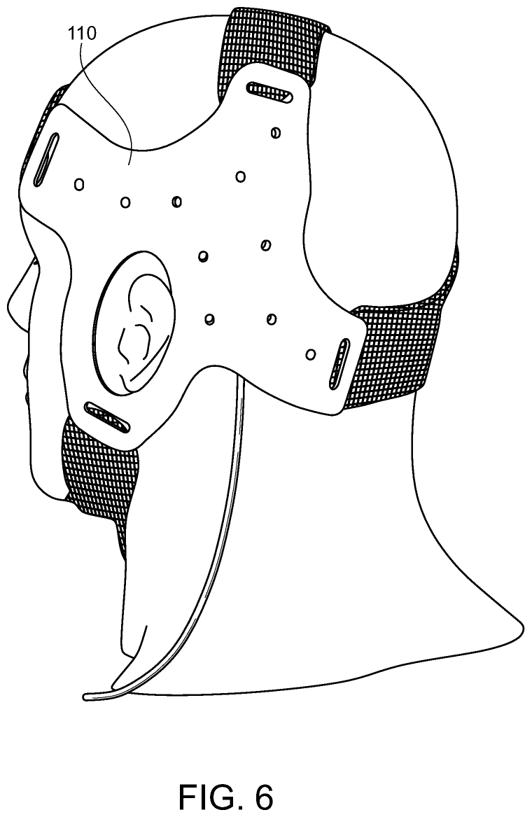

With reference to FIGS. 2, 5, and 6, the wearable headgear holding device 200 (FIG. 2) includes a headgear or strap portion or holding textile 216. The holding textile 216 can include at least one of, some of, or all of, the following straps: a neck strap 238a, a strap 238b configured to extend over a top portion of the head, a forehead strap 238c, and a strap 238d for a back of the head. The neck strap 238a and top strap 238b can be optional, and it will be appreciated that other types and configurations of straps can be used. The at least one pad 110 (FIG. 1) can include at least one elongated arm 140a-140d configured to permit attachment to at least one of the neck strap 238a (FIG. 2), the strap 238b (FIG. 2) configured to extend over the top portion of the head, the forehead strap 238c (FIG. 2), and the strap 238d (FIG. 2) for the back of the head. The holding textile 216 can form an opening 242 for an ear of the head on opposite sides. The holding textile 216 can be formed of a fitting, extendable, resistant, breathable, and washable material, such as a webbing material. In some embodiments, lower arm 140a of the pad portion or the at least one pad 110 can be aligned and attached to the neck strap 238a, upper arm 140b can be aligned and attached to strap 238b for the top portion of the head, front arm 140c can be aligned and attached to forehead strap 238c, and back arm 140d can be aligned and attached to strap 238d for the back of the head. Attachment of the arms 140a-140d in alignment to the appropriate respective straps 238a-238d can align and/or position the cavity 114 and neural implant head-piece 205 in the desired proper location of the user's head. The holding textile 216 can extend around and cover the outer surface, outer facing side or outer face of the pad portion or the at least one pad 110. As can be seen in FIG. 2, the holding textile 216 can bend the arms 140a-140d inwardly towards each other in a curved manner. When the at least one pad 110 is held in place by the holding textile 216, the mesas 122 can conform to the shape of the head and spread out the force or pressure applied by the holding textile 216 over a large padded surface area for more comfort, instead of over only the neural implant head-piece 205. The inner facing surface of the neural implant head-piece 205 that is positioned next to the head can be flush with the inner facing surfaces of the tops of the surrounding mesas 122a-122d, or in other embodiments, can slightly protrude or can be slightly recessed.

In addition, FIG. 7 shows that a rear portion 732, or other portion 734, of the neural implant head-piece 205 may be shaped to fit within a pocket or other opening within the at least one pad 110 (FIG. 1), such as in cavity 114 (FIG. 1).

The neural implant head-piece can be configured to be mounted to the exterior head in any of a variety of different ways. In one example, the neural implant head-piece is mounted using adhesives; in another example, it is mounted magnetically, for example using one or more magnetic pole pieces attached to the head-piece and being attracted to magnetic pole pieces on or within the head; in another example, the neural head-piece need not be affixed to the head, but merely may be partly or wholly contained loosely, and yet securely, in the cavity 114 (FIG. 1) by being trapped between the pad 110 and the exterior of the head. Where the neural implant head-piece is magnetically mounted to the exterior of the head, the cavity 114 can be formed to have a height clearance (i.e. vertical distance) between a bottom surface 144 (see FIG. 1) of the cavity 114 of the at least one pad 110 and the rear portion 732 (FIG. 7) of the neural implant headpiece that is no larger than a distance of magnetic misalignment of the neural implant headpiece, such as 12 mm or less of height clearance. Such a height clearance may also be used regardless of whether the neural implant head-piece is magnetically mounted. In addition, regardless of whether the neural implant head-piece is magnetically mounted, a side clearance between the at least one pad and the neural implant headpiece can, for example, be 5 mm or less of clearance between an inside perimeter 146 (FIG. 1) of the cavity 114 (FIG. 1) and an outside perimeter of the neural implant headpiece (e.g., an outside perimeter of rear portion 732 of FIG. 7).

The neural implant headpiece can, for example, be one that is worn for treatment, alleviation, or monitoring of any of a variety of different conditions, including neurological and psychiatric disorders and conditions, such as epilepsy, dementia, Alzheimer's disease, Parkinson's disease, and others. The wearable holding device can, for example, be used by people with neural implants that are spending at least some of their day in bed, such as paralyzed people, elderly people, comatose patients, locked-in patients, and people with other neural conditions that affect mobility. The implant can, for example, partly or wholly within the skull, and can be a neural implant, cochlear implant, or other suitable implant.

While example embodiments have been particularly shown and described, it will be understood by those skilled in the art that various changes in form and details may be made therein without departing from the scope of the embodiments encompassed by the appended claims. Although the pad portion 110 has been shown to be mounted to the left side of the head, in other embodiments, mounting can be on the right side of the head.

1. A wearable holding device for a neural implant head-piece, the wearable holding device comprising:
at least one pad surrounding at least part of the neural implant head-piece, the at least part of the neural implant head-piece being configured to be mounted to an exterior of a head, the at least one pad forming a cavity configured to receive and cover the at least part of the neural implant head-piece and to maintain stability of the neural implant head-piece relative to the head; and
a holding textile configured to hold the at least one pad forming the cavity to the head.

2. The wearable holding device of claim 1, wherein the at least part of the neural implant head-piece is configured to communicate at least one of power and data to or from at least one of a power source and a data source.

3. The wearable holding device of claim 1, wherein the at least part of the neural implant head-piece comprises a battery.

4. The wearable holding device of claim 1, wherein the at least one pad is configured to deform in shape to follow a topography of the head.

5. The wearable holding device of claim 1, wherein the at least one pad comprises a plurality of padding mesas extending to a height from a surface of the at least one pad, the plurality of padding mesas being separated from each other by padding gaps configured to permit the plurality of padding mesas to be moved closer to each other or further away from each other as the at least one pad is deformed by pressure on the head.

6. The wearable holding device of claim 5, wherein the cavity configured to receive and cover the at least a part of the neural implant head-piece is formed by at least some of the plurality of padding mesas.

7. The wearable holding device of claim 1, wherein the at least part of the neural implant head-piece is configured to communicate at least one of power and data to or from at least one of a power source and a data source, and wherein the at least one pad forms a cord opening for at least one cord carrying the at least one of power and data to or from the at least one of the power source and the data source.

8. The wearable holding device of claim 7, further comprising a plurality of padding mesas separated from each other by padding gaps, and wherein the cord opening is formed by at least some of the plurality of padding mesas.

9. The wearable holding device of claim 1, wherein the at least one pad forms an opening for an ear of the head.

10. The wearable holding device of claim 1, wherein the cavity configured to receive and cover the at least a part of the neural implant head-piece is formed to be positioned over at least one of a parietal bone of the head, a temporal bone of the head, a mastoid process of the head, and an occipital bone of the head, when the holding textile holds the at least one pad forming the cavity to the head.

11. The wearable holding device of claim 1, wherein the at least one pad forms at least one textile opening configured to permit attaching of the holding textile to the at least one pad.

12. The wearable holding device of claim 1, wherein the holding textile comprises at least one of: a neck strap, a strap configured to extend over a top portion of the head, a forehead strap, and a strap for a back of the head.

13. The wearable holding device of claim 12, wherein the holding textile comprises the neck strap, the strap configured to extend over the top portion of the head, the forehead strap, and the strap for a back of the head.

14. The wearable holding device of claim 12, wherein the at least one pad comprises at least one elongated arm configured to permit attachment to at least one of the neck strap, the strap configured to extend over the top portion of the head, the forehead strap, and the strap for the back of the head.

15. The wearable holding device of claim 1, wherein the holding textile forms an opening for an ear of the head.

16. The wearable holding device of claim 1, wherein the neural implant head-piece is configured to be magnetically mounted to the exterior of the head, and wherein the cavity configured to receive and cover the at least a part of the neural implant head-piece is formed to comprise a surrounding clearance between the at least one pad and the neural implant headpiece that is no larger than a distance of magnetic misalignment of the neural implant headpiece.

17. The wearable holding device of claim 1, wherein a surrounding clearance between the at least one pad and the neural implant headpiece comprises 5 mm or less of clearance between the at least one pad and a side perimeter of the neural implant headpiece.

18. The wearable holding device of claim 16, wherein the surrounding clearance comprises 12 mm or less of height clearance between a top portion of the neural implant headpiece and the at least one pad.

19. The wearable holding device of claim 1, wherein the at least one pad comprises a breathable foam.

20. The wearable holding device of claim 1, wherein the at least one pad comprises a closed cell foam.

21. The wearable holding device of claim 20, wherein the closed cell foam comprises at least one of an ethylene-vinyl acetate (EVA) foam and a polyurethane foam.

22. The wearable holding device of claim 1, wherein the holding textile comprises a webbing material.

23. A method of wearing a neural implant head-piece comprising:
surrounding at least part of the neural implant head-piece with at least one pad of a wearable holding device, the at least part of the neural implant head-piece being mounted to an exterior of a head, the at least one pad forming a cavity receiving and covering the at least part of the neural implant head-piece and to maintain stability of the neural implant head-piece relative to the head; and
holding the at least one pad forming the cavity to the head with a holding textile.

24. The method of claim 23, further comprising deforming the shape of the at least one pad to follow the topography of the head.

25. The method of claim 24, wherein the at least one pad comprises a plurality of padding mesas extending to a height from a surface of the at least one pad, the plurality of padding mesas being separated from each other by padding gaps configured to permit the plurality of padding mesas to be moved closer to each other or further away from each other as the at least one pad is deformed by pressure on the head.

26. The method of claim 25, wherein the cavity configured to receive and cover the at least part of the neural implant head-piece is formed by at least some of the plurality of padding mesas.

27. The method of claim 23, wherein the at least one pad forms an opening for an ear of the head.
